(19) United States
(12) Patent Application Publication
(10) Pub. No.: US 2008/0319358 A1
(43) Pub. Date: Dec. 25, 2008

(54) Massage Tool

(75) Inventor: Tzn-Yung Lai, Taiwan (CN)

Correspondence Address: BROWDY AND NEIMARK, P.L.L.C.;624 NINTH STREET, NW, SUITE 300, WASHINGTON, DC, 20001-5303, US

(73) Assignee: CHUEN JEOU DAY ENTERPRISE CO., LTD, Taiwan (CN)

(21) Appl. No.: 12/095,916

(22) PCT Filed: Dec. 1, 2006
(86) PCT No.: PCT/CN06/03245

§ 371 Date: Jun. 2, 2008

(30) Foreign Application Priority Data
Dec. 2, 2005 (CN) 200520133600.1
Feb. 13, 2006 (CN) 200620001559.7
Nov. 30, 2006 (CN) 200620160590.5

(51) Int. Cl. A61F 2/06

(52) U.S. Cl. 601/134; 601/135; 601/137

(57) ABSTRACT

A massage tool includes a handle, which has a cylinder provided for holding, a barrel hole going through the cylinder; a first barrel, which has a barrel body inserted into the barrel hole of the handle, a chamber formed concaved on the barrel body, a fitting hole opened downwards from the bottom of the chamber; a toe, which has a shoulder received in the chamber of the first barrel; a second barrel, which has a barrel body inserted into the barrel hole of the handle; the massage tool which assembled via above structure is convenient for use and holding; further, the massage tool can includes a skirt, which has a skirt body mounted on the handle, the second barrel and the skirt mounted at least two fingers or a palm; and a magnet group received in the chamber of the first barrel; thereby, the invention can assembled variety of massage tool, the magnet group generating magnetic field improve human body blood circulation, and the toe or the finger to proceed pressing.

### FIELD OF THE INVENTION

The present invention relates to tools of massage and more specifically, to a massage tool.

### DESCRIPTION OF THE RELATED ART

The structure of prior massage tool, commonly comprise a cylinder body, two caps, a toe, and a plurality of magnets, wherein the cylinder body is provided mounting of each cap, the toe mounted on one of the two caps, each magnet can be received inside the chamber which formed by each cap assembled with the cylinder body, then get the massage tool.

But there are many problems in manufacturing method of the prior tool, and the structure is complicated, causing waste time of assembly and higher the cost of the production, and the user is not willing to buy.

### SUMMARY OF THE INVENTION

The main object of the massage tool of the invention, which uses a new assembly of barrel and handle, to simplify the assembly structure, and easier the assemble of the toe and magnet.

Another object of the massage tool of the invention, wherein the assembly of the parts is convenient, and the assembled structure is stronger.

Still another object of the massage tool of the invention, which has a plurality of magnets, when using the massage tool to massaging, still has the magnetic effect produced by the magnets.

To achieve these and other objects of the invention, the massage tool of the invention comprises a handle, which has a cylinder provided for holding, a barrel hole going through the cylinder, a first rim formed at end of the cylinder; a first barrel, which has a barrel body inserted into the barrel hole of the handle, a chamber formed concaved on the barrel body, a barrel end connected to the barrel body, a rim stopped against the first rim of the handle, a fitting hole opened from bottom of the chamber through the barrel end; a toe, which has a shoulder received inside the chamber of the first barrel, a toe post inserted to the fitting hole of the first barrel, a toe head extended outside the barrel end of the first barrel; a second barrel, which has a barrel body inserted into the barrel hole of the handle, a barrel end connected to the barrel body; a magnet received in the chamber of the first barrel.

In the technical solution of above invention, wherein the quantity of the magnets is at least two, each magnet being arranged by different pole between the first barrel and the second barrel.

In the technical solution of above invention, wherein the toe further threads with the first barrel.

In the technical solution of above invention, further comprises a second magnet, the toe having a chamber for receiving the second magnet.

In the technical solution of above invention, further comprises a first spring biased between the toe and first barrel.

In the technical solution of above invention, further comprises a second spring biased between the magnet and second barrel.

In the technical solution of above invention, wherein the first barrel is threaded with the second barrel.

In the technical solution of above invention, wherein the second barrel further has a chamber for receiving the magnet.

In the technical solution of above invention, wherein the handle has a second rim, the second barrel has a rim for stopping against the second rim of the handle.

Another massage tool of the invention comprises a handle, a skirt, at least two fingers, and at least two magnets, the skirt mounted on the handle, the finger mounted on the skirt, each finger has a chamber, at least one magnet received in each chamber of the finger; thereby, the invention not only can using the finger to proceed pressing, but also the magnets generating magnetic field to achieve the object of health care.

In the technical solution of above invention, wherein the fingers are welded onto the skirt.

In the technical solution of above invention, wherein the skirt has a plurality of joints, each joint has thread, each finger has an joint, and each joint has a thread, the joint of the finger threaded to the joint of the skirt.

In the technical solution of above invention, wherein the handle has a cap, the cap is beset at bottom of the handle.

In the technical solution of above invention, wherein the cap is connected to the handle by friction welding.

Still another massage tool of the invention comprises a handle, which has a cylinder provided for holding, a barrel hole going through the cylinder; a first barrel, which has a barrel body inserted into the barrel hole of the handle, a chamber formed concaved on the barrel body, a fitting hole opened downwards from the bottom of the chamber; a toe, which has a shoulder received in the chamber of the first barrel; a second barrel, which has a barrel body inserted into the barrel hole of the handle, a first outer thread formed at the bottom of the barrel body and threaded to the thread portion of the handle, a barrel end connected to the barrel body, a second outer thread formed on the barrel end; at least one first magnet received in the first barrel and the chamber of second barrel; a skirt, which has a skirt body mounted on the handle, a thread hole formed on middle of the skirt body and threaded to the second outer thread of the second barrel; and at least two fingers connected with the skirt; thereby, the invention can using the finger to proceed pressing, and the assemble strength of the elements is more stable.

In the technical solution of above invention, further comprises at least one second magnet received in the chamber of the toe.

In the technical solution of above invention, further comprises at least two third magnets received in each chamber of the finger.

In the technical solution of above invention, wherein the skirt has at least one joint, each joint has thread; each finger has an joint which has thread, each joint of the finger each threaded to each joint of the skirt.

In the technical solution of above invention, wherein each joint of the finger is threaded to the second outer thread of the barrel end of the second barrel.

In the technical solution of above invention, wherein the handle further has a second rim opposite to the first rim, the skirt is welded on the second rim of the handle.

In the technical solution of above invention, wherein each finger is welded onto the skirt.

A fourth massage tool of the invention comprises a handle having a cylinder provided for holding, a barrel hole going through the cylinder, a first rim formed at end of the cylinder, a thread portion formed opposite the first rim; a first barrel having a barrel body inserted into the barrel hole of the handle, a chamber formed concaved on the barrel body, a rim stopped against the first rim of the handle, a fitting hole opened downwards from the bottom of the chamber; a toe having a shoulder received in the chamber of the first barrel, a toe post inserted to the fitting hole of the first barrel, a toe head formed on the top of the toe post; a second barrel having a barrel body inserted into the barrel hole of the handle, a thread portion formed on the barrel body and threaded to the thread portion of the handle, a fitting hole formed on the barrel end; at least one first magnet received in the chamber of the first barrel; a skirt having a skirt body mounted on the handle, a palm jointing slot formed at middle of the skirt body; and a palm having a palm plate correspondingly mounted on the palm jointing slot of the skirt, a stem formed at the bottom of the palm plate and fitted to the fitting hole of the second barrel.

In the technical solution of above invention, the handle has a second rim opposite to the first rim, the skirt is welded on the second rim.

In the technical solution of above invention, the palm is welded on the skirt.

The advantage of the invention: The massage tool of the invention, which uses a new assembly of barrel and handle, to simplify the assembly structure, and easier the assemble of the toe and magnet. The massage tool of the invention, wherein the assembly of the part is convenient, and the assembled structure is stronger. The massage tool of the invention, which has a plurality of magnets, when using the massage tool to massaging, still has the magnetic effect produced by the magnets.

The massage tool of the invention not only can using the finger to solve the problem of aches, but also the magnets further improved blood circulation of human body, to achieve the object of health care.

### BRIEF DESCRIPTION OF THE DRAWINGS

FIG. 1 shows an elevational assembly view of the first embodiment of the invention.

FIG. 2 shows an elevational explode view of the first embodiment of the invention.

FIG. 3 shows a plane explode view the first embodiment of the invention.

FIG. 4 shows an assembly sectional view of the first embodiment of the invention.

FIG. 5 shows an assembly sectional view of a second embodiment of the invention.

FIG. 6 shows an assembly sectional view of a third embodiment of the invention.

FIG. 7 shows an assembly sectional view of a fourth embodiment of the invention.

FIG. 8 shows an assembly sectional view of a fifth embodiment of the invention.

FIG. 9 shows an assembly sectional view of a sixth embodiment of the invention.

FIG. 10 shows an elevational view of a seventh embodiment of the invention.

FIG. 11 shows an elevational explode view of the seventh embodiment of the invention.

FIG. 12 shows a sectional view of the seventh embodiment of the invention.

FIG. 13 shows a sectional view of an eighth embodiment of the invention.

FIG. 14 shows an elevational explode view of a ninth embodiment of the invention.

FIG. 15 shows a sectional view of the ninth embodiment of the invention.

FIG. 16 shows a partial elevational explode view of a tenth embodiment of the invention.

FIG. 17 shows a sectional view of the tenth embodiment of the invention.

### DETAILED DESCRIPTION OF THE INVENTION

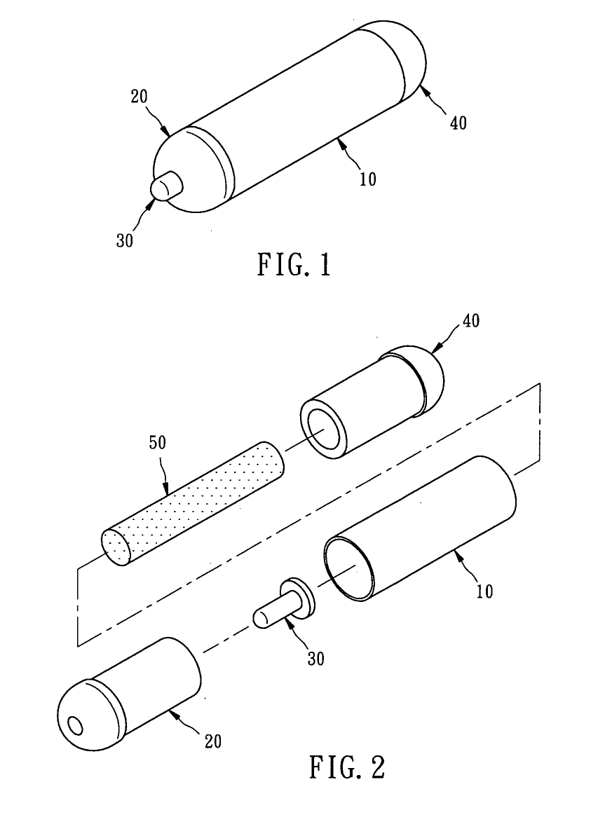

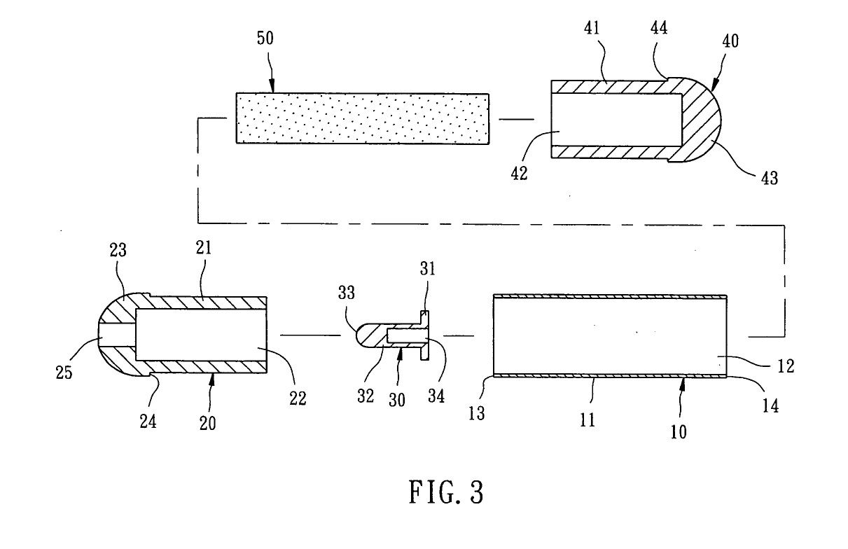

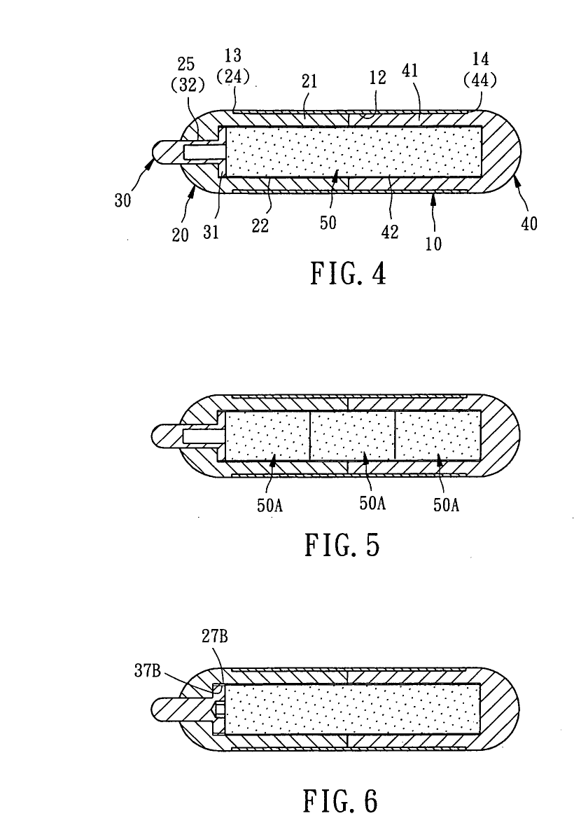

Please refer FIG. 1 to FIG. 4, a massage tool of the first embodiment of the invention comprises the follows:

A handle 10 has a cylinder 11 being held by hand, a barrel hole 12 going through the cylinder 11, a first rim 13 and a second rim 14 formed at two sides of the cylinder 11 respectively.

A first barrel 20 has a barrel body 21 inserted into the barrel hole 12 of the handle 10, a chamber 22 concaved formed from the first barrel 20 upwards and formed of a loop section, a barrel end 23 formed on the barrel body 21, a rim 24 stopped against the first rim 13 of the handle 10, a fitting hole 25 opened through the chamber 22 bottom.

A toe 30 has a shoulder 31, a toe post 32 being inserted through the fitting hole 25 of the first barrel 20, the toe post 32 formed a semi-spherical shape at top, a toe head 33 protruded from the toe 30, a chamber 34 formed at the bottom of the toe 30, the chamber 34 concaved formed from the toe 30 bottom upwards and formed of a loop section.

A second barrel 40 has a barrel body 41 inserted into the barrel hole 12 of the handle 10, a chamber 42 concaved formed from the second barrel 40 upwards and formed of a loop section, a barrel end 43 formed on the barrel body 41, a rim 44 stopped against the second rim 14 of the handle 10.

A magnet 50 has two ends received in the chamber 22 of the first barrel 20 and the chamber 42 of the second barrel 40 respectively, the section shape and size of the magnet 50 substantially same as the chamber 22 and 42.

By above structure, the handle 10 can be hold by user, let the toe post 32 of the toe 30 pressed to desired portion, because the toe post 32 and the magnet 50 conducted, the magnet 50 then generated a magnet field, and being surrounded around the toe post 32 and return to the second barrel 40, the magnet field line of the massage tool then achieving the both massage effect and magnetic function.

The invention further has the following embodiments:

First, as shown in FIG. 5, shows the second embodiment of the invention; wherein the quantity of the magnets 50A is three, each magnet 50A arranged by different pole adjacent to each other.

Further, as shown in FIG. 6, the third embodiment of the invention is substantially same as above embodiment, wherein the first barrel formed an inner thread 27B, the toe formed an outer thread 37B threaded to the inner thread 27B of the first barrel, then the toe can thread with the first barrel.

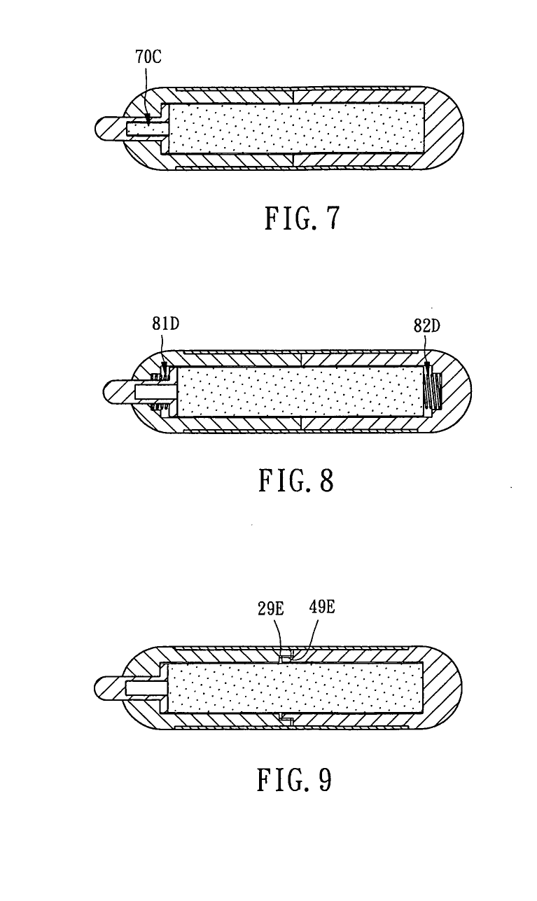

As shown in FIG. 7, the fourth embodiment of the invention further comprises a second magnet 70C received inside the chamber of the toe, the second magnet 70C also providing the magnetic effect.

As shown in FIG. 8, the massage tool of the fifth embodiment of the invention further comprises a first spring 81D fitted to the toe post of the toe, and a second spring 82D mounted in the second barrel and biased against the magnet; thereby, the first spring 81D and the second spring 82D providing the massage tool with a damping function to preventing shock.

As shown in FIG. 9, the sixth embodiment of the invention, wherein the first barrel has an inner thread 29E, the second barrel has an outer thread 49E, the first barrel and the second barrel assembled via the inner thread 29E and the outer thread 49E threaded to each other.

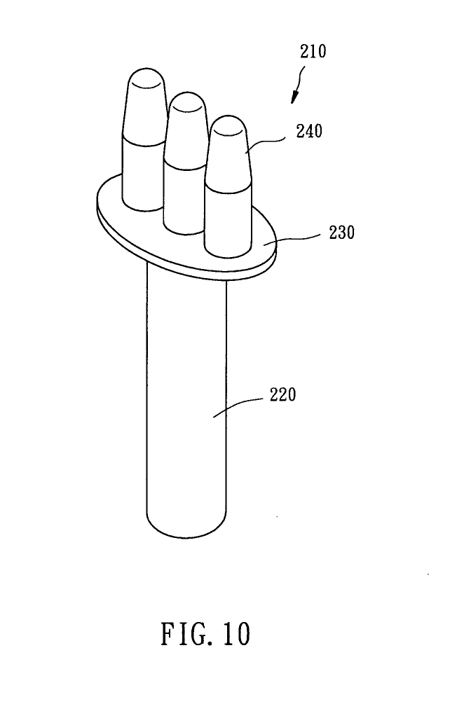

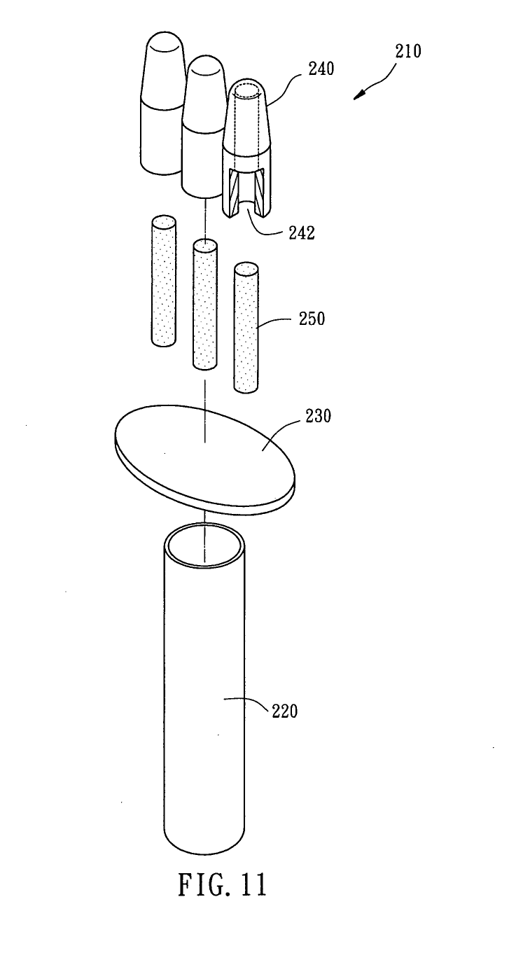

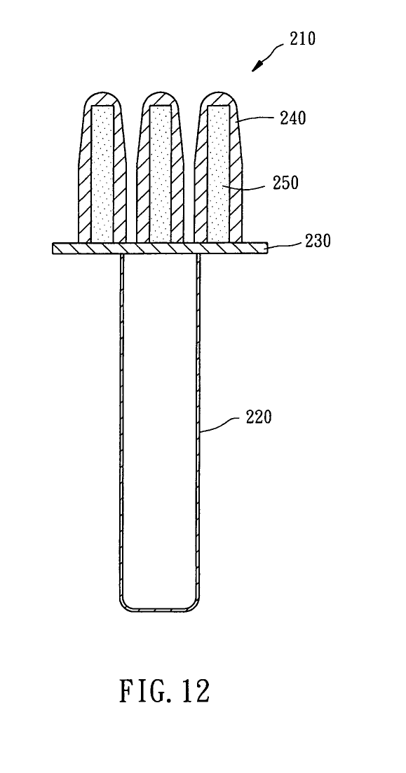

Please refer FIG. 10 to FIG. 12, the massage tool 210 of the seventh embodiment of the invention comprises a handle 220, a skirt 230, three fingers 240, and three magnets 250.

The handle 220 is a metal hollow cylinder, which is provided for holding.

The skirt 230 is a metal circle skirt body, the skirt 230 is welded on the top of the handle 220.

The fingers 240 each is a metal pin and having a conic round end, the fingers 240 are welded onto top of the skirt 230 and linear arrayed, each finger 240 has a chamber 242.

The magnets 250 are formed of cylindrical shape, the magnets 250 are assembled into the chamber 242 of the finger 240.

By above structure, when user using the massage tool 210, the handle 220 can be hold by single hand, let the finger 240 pressing anywhere of the human body to proceed massaging, when the finger 240 to proceed pressing, the magnets 250 generating a magnetic field, which improve blood circulation of human body, to recover health of human body and enhance energy.

Thereby, the invention not only can using the finger 240 to solve the problem of aches, but also the magnets 250 further improved blood circulation of human body, to achieve the object of health care.

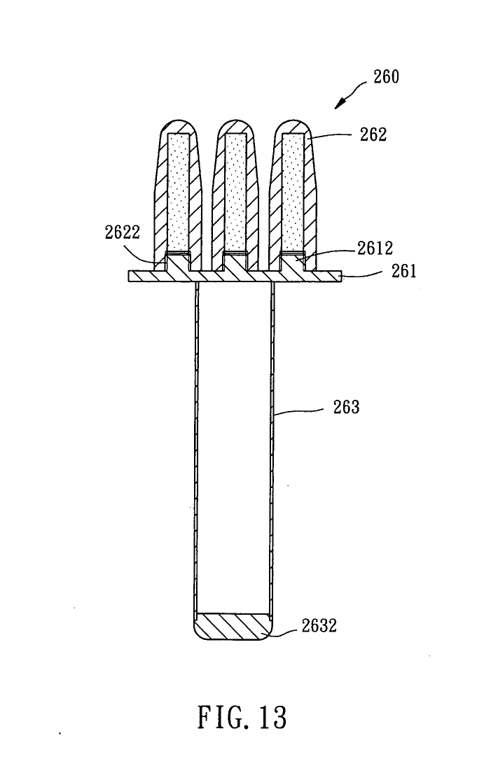

Please refer FIG. 13, the massage tool 260 of the eighth embodiment of the invention is substantially same as the seventh embodiment, the differences are:

The skirt 261 has a plurality of skirt-to-finger joints 2612 on the top, each skirt-to-finger joint 2612 has thread, the finger 262 also has a finger-to-skirt joint 2622, the finger-to-skirt joint 2622 also has thread, the finger-to-skirt joint 2622 of the finger 262 is threaded to the skirt-to-finger joint 2612 of the skirt 261; moreover, the handle 263 has a cap 2632, the cap 2632 is beset at the bottom of the handle 263, or friction welded to the handle 263.

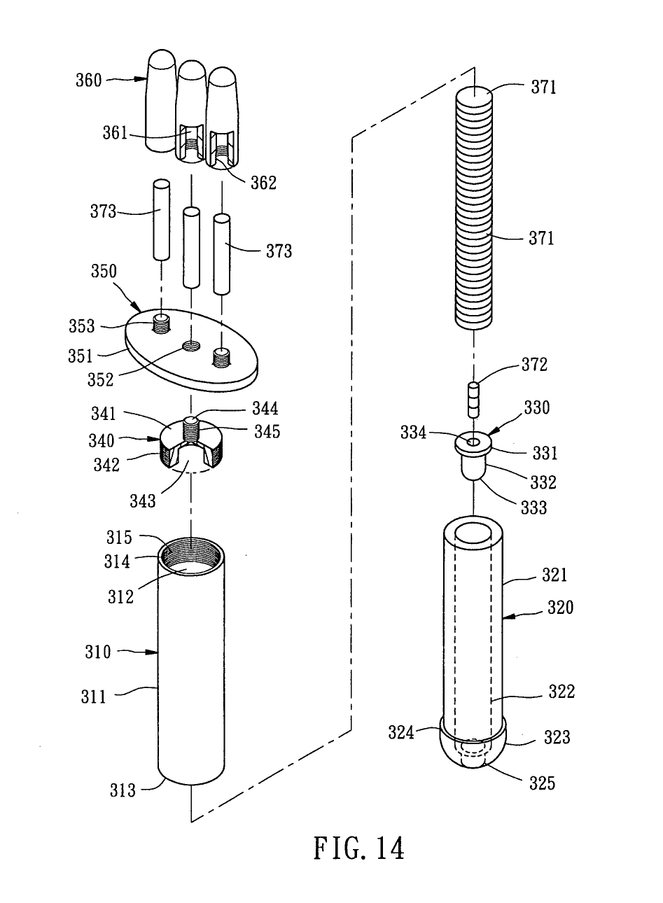

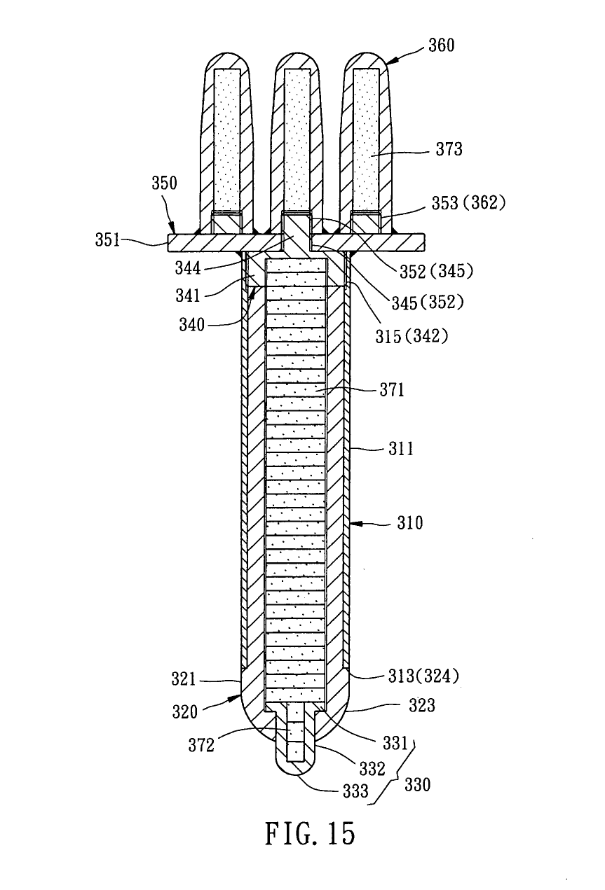

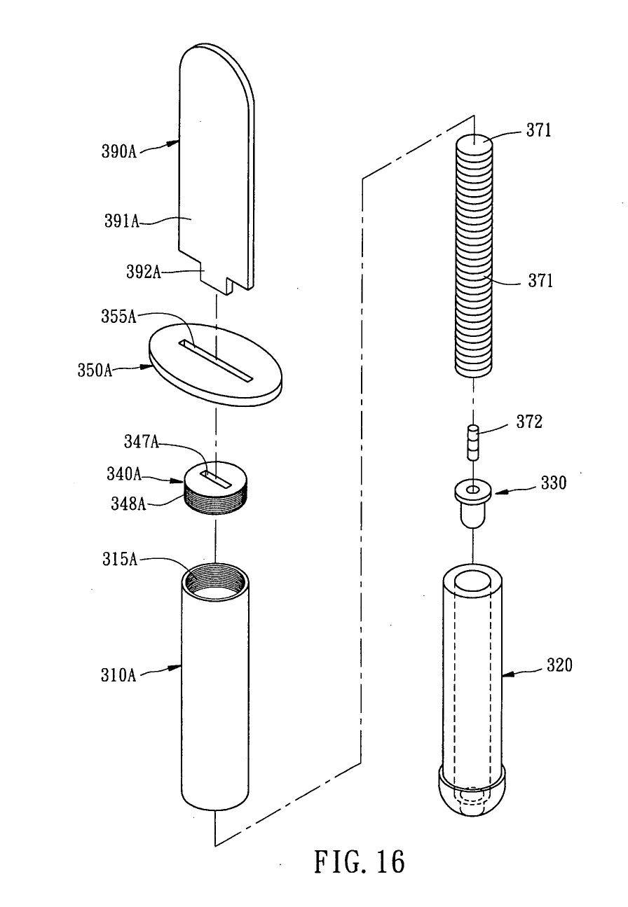

Please refer FIG. 14 and FIG. 15, the massage tool of the ninth embodiment of the invention comprises the follows:

A handle 310 has a cylinder 311 being held by hand, a barrel hole 312 going through the cylinder 311, a first rim 313 and a second rim 314 formed at two sides the cylinder 311 respectively, a thread portion 315 formed on the cylinder 311.

A first barrel 320 has a barrel body 321 inserted into the barrel hole 312 of the handle 310, a chamber 322 concaved formed from the first barrel 320 upwards and formed of a loop section, a barrel end 323 formed on the barrel body 321, a rim 324 stopped against the first rim 313 of the handle 310, a fitting hole 325 formed at bottom of the chamber 322.

A toe 330 has a shoulder 331, a toe post 332 being inserted through the fitting hole 325 of the first barrel 320, a toe head formed of a semi-spherical shape 333 protruded from the free end of the toe post 332, a chamber 334 formed at bottom of the toe 330, the chamber 334 concaved formed from the toe 330 bottom upwards and formed of a loop section.

A second barrel 340 has a barrel body 341, a first outer thread 342 formed at the bottom of the barrel body 341 and threaded to the thread portion 315 of the handle 310, a chamber 343 concaved formed from the second barrel 340 upwards and formed a loop section, a barrel end 344 protruded from the top of the barrel body 341, a second outer thread 345 formed on the barrel end 344.

A skirt 350 has a skirt body 351 formed of an oval shape and provided for weld mounting of the second rim 314 of the handle 310, a thread hole 352 formed at middle of the skirt body 351 and threaded to the second outer thread 345 of the second barrel 340, two skirt-to-finger joints 353 each mounted on two sides of the skirt body 351 and formed a thread.

Three fingers 360, which is made by metal and formed conic at front, each finger 360 each concaved formed a chamber 361, a finger-to-skirt joint 362 which is thread formed at the bottom of the chamber 361, the finger-to-skirt joint 362 threaded to the two skirt-to-finger joints 353 of the skirt 350 and second outer thread 345 of the second barrel 340, each finger 360 threaded and weld on the skirt 350.

A magnet group, which has a plurality of first magnets 371 received in the chamber 322 and 343 of the first barrel 320 and the second barrel 340, the section shape and size of the first magnet 371 substantially same as the chamber 322 and 343; a plurality of second magnets 372 received in the chamber 334 of the toe 330, the section shape and size of the second magnet 372 substantially same as the chamber 334; three magnets 373 of cylindrical shape installed inside the chamber 361 of the finger 360.

Via the structure described above, when user using the massage tool, holding the handle 310 by single hand, let the toe 330 or each finger 360 pressing the vulnerable point or the portion of aches to proceed pressing.

Wherein the handle 310, skirt 350 and each finger 360 further welded to each other, thereby promoting the strength of the structure.

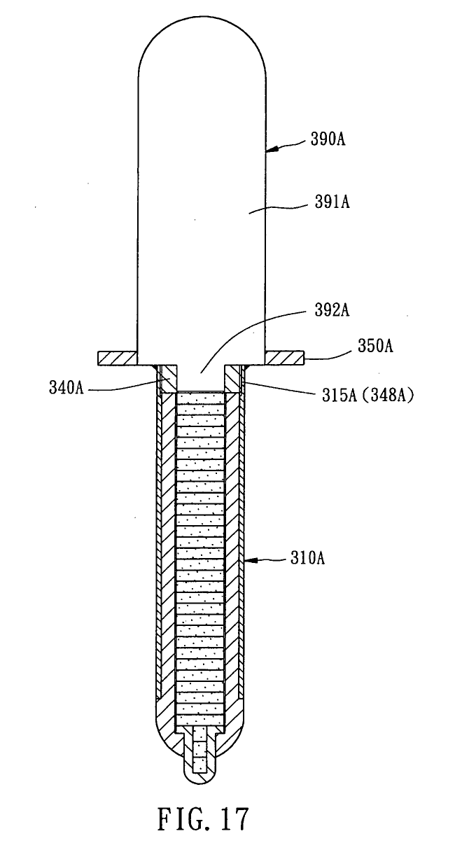

Please refer FIG. 16 and FIG. 17, the tenth embodiment of the invention is substantially same as the first embodiment, the differences are:

The second barrel 340A opened through a stem hole 347A, a thread portion 348A formed on the second barrel 340A and threaded to the thread portion 315A of the handle 310A; the skirt 350A has a palm jointing slot 355A; a palm 390A has a palm plate 391A of long shape, and a stem 392A formed at the bottom of the palm plate 391A, the palm plate 391A fitted to the palm jointing slot 355A of the skirt 350A, the stem 392A fitted to the stem hole 347A of the second barrel 340A, the handle 310A weld on the skirt 350A; therefore, the palm 390A capable of providing different massaging way.

Concluding the aforesaid embodiments, the massage tool of the invention has the characters of follows:

1. The massage tool of the invention, which uses a new assembly of barrel and handle, to simplify the assembly structure, and easier the assemble of the toe and magnet.

2. The massage tool of the invention, wherein the assembly of the part is convenient, and the assembled structure is stronger.

3. The massage tool of the invention, which has a plurality of magnets, when using the massage tool to massaging, still has the magnetic effect produced by the magnets.

### INDUSTRIAL APPLICABILITY

The massage tool of the invention not only can using the finger to solve the problem of aches, but also the magnets further improved blood circulation of human body, to achieve the object of health care.

What is claimed is:

1. A massage tool comprising:
a handle having a cylinder provided for holding, a barrel hole going through the cylinder, a first rim formed at end of the cylinder;
a first barrel having a barrel body inserted into the barrel hole of the handle, a chamber formed concaved on the barrel body, a barrel end connected to the barrel body, a rim stopped against the first rim of the handle, a fitting hole opened from bottom of the chamber through the barrel end;
a toe having a shoulder received inside the chamber of the first barrel, a toe post inserted to the fitting hole of the first barrel, a toe head extended outside the barrel end of the first barrel;
a second barrel having a barrel body inserted into the barrel hole of the handle, a barrel end connected to the barrel body;
a magnet received in the chamber of the first barrel.

2. The massage tool as claimed in claim 1, wherein the quantity of the magnets is at least two, each magnet being arranged by different pole between the first barrel and the second barrel.

3. The massage tool as claimed in claim 1, wherein the toe further threads with the first barrel.

4. The massage tool as claimed in claim 1, further comprising a second magnet, the toe having a chamber for receiving the second magnet.

5. The massage tool as claimed in claim 1, further comprising a first spring biased between the toe and first barrel.

6. The massage tool as claimed in claim 5, further comprising a second spring biased between the magnet and second barrel.

7. The massage tool as claimed in claim 1, wherein the first barrel is threaded with the second barrel.

8. The massage tool as claimed in claim 1, wherein the second barrel further has a chamber for receiving the magnet.

9. The massage tool as claimed in claim 1, wherein the handle has a second rim, the second barrel has a rim for stopping against the second rim of the handle.

10. A massage tool comprising:
a handle;
a skirt connected to the handle;
at least two fingers connected with the skirt, each finger having a chamber; and
at least two magnets, each magnet received in each chamber of the finger.

11. The massage tool as claimed in claim 10, wherein the fingers are welded onto the skirt.

12. The massage tool as claimed in claim 10, wherein the skirt has a plurality of joints, each joint has thread, each finger has an joint, and each joint has a thread, the joint of the finger threaded to the joint of the skirt.

13. The massage tool as claimed in claim 10, wherein the handle has a cap, the cap is beset at bottom of the handle.

14. The massage tool as claimed in claim 13, wherein the cap is connected to the handle by friction welding.

15. A massage tool comprising:
a handle having a cylinder provided for holding, a barrel hole going through the cylinder, a first rim formed at end of the cylinder, a thread portion formed opposite the first rim;
a first barrel having a barrel body inserted into the barrel hole of the handle, a chamber formed concaved on the barrel body, a rim stopped against the first rim of the handle, a fitting hole opened downwards from the bottom of the chamber;
a toe having a shoulder received in the chamber of the first barrel, a toe post inserted to the fitting hole of the first barrel, a toe head formed on the top of the toe post;
a second barrel having a barrel body inserted into the barrel hole of the handle, a first outer thread formed at the bottom of the barrel body and threaded to the thread portion of the handle, a barrel end connected to the barrel body, a second outer thread formed on the barrel end;
at least one first magnet received in the first barrel and the chamber of second barrel;
a skirt having a skirt body mounted on the handle, a thread hole formed on middle of the skirt body and threaded to the second outer thread of the second barrel; and
at least two fingers connected with the skirt, each finger having a chamber.

16. The massage tool as claimed in claim 15, further comprising at least one second magnet received in the chamber of the toe.

17. The massage tool as claimed in claim 15, further comprising at least two third magnets received in each chamber of the finger.

18. The massage tool as claimed in claim 15, wherein the skirt has at least one joint, each joint has thread; each finger has an joint which has thread, each joint of the finger each threaded to each joint of the skirt.

19. The massage tool as claimed in claim 18, wherein each joint of the finger is threaded to the second outer thread of the barrel end of the second barrel.

20. The massage tool as claimed in claim 15, wherein the handle further has a second rim opposite to the first rim, the skirt is welded on the second rim of the handle.

21. The massage tool as claimed in claim 15, wherein each finger is welded onto the skirt.

22. A massage tool comprising:
a handle having a cylinder provided for holding, a barrel hole going through the cylinder, a first rim formed at end of the cylinder, a thread portion formed opposite the first rim;
a first barrel having a barrel body inserted into the barrel hole of the handle, a chamber formed concaved on the barrel body, a rim stopped against the first rim of the handle, a fitting hole opened downwards from the bottom of the chamber;
a toe having a shoulder received in the chamber of the first barrel, a toe post inserted to the fitting hole of the first barrel, a toe head formed on the top of the toe post;
a second barrel having a barrel body inserted into the barrel hole of the handle, a thread portion formed on the barrel body and threaded to the thread portion of the handle, a fitting hole formed on the barrel end;
at least one first magnet received in the chamber of the first barrel;
a skirt having a skirt body mounted on the handle, a palm jointing slot formed at middle of the skirt body; and
a palm having a palm plate correspondingly mounted on the palm jointing slot of the skirt, a stem formed at the bottom of the palm plate and fitted to the fitting hole of the second barrel.

23. The massage tool as claimed in claim 22, the handle has a second rim opposite to the first rim, the skirt is welded on the second rim.

24. The massage tool as claimed in claim 22, the palm is welded on the skirt.
